(19) United States
(12) Patent Application Publication
(10) Pub. No.: US 2024/0252713 A1
(43) Pub. Date: Aug. 1, 2024

(54) ADHESIVE, WOUND DRESSING MATERIAL, ANTI-ADHESION MATERIAL, HEMOSTATIC MATERIAL, SEALANT, AND SPRAY KIT

(71) Applicant: NATIONAL INSTITUTE FOR MATERIALS SCIENCE, Tsukuba-shi, Ibaraki (JP)

(72) Inventor: Tetsushi TAGUCHI, Tsukuba-shi (JP)

(73) Assignee: NATIONAL INSTITUTE FOR MATERIALS SCIENCE, Tsukuba-shi, Ibaraki (JP)

(21) Appl. No.: 18/577,332

(22) PCT Filed: Apr. 19, 2022
(86) PCT No.: PCT/JP2022/018105

§ 371 Date: Jan. 8, 2024

(30) Foreign Application Priority Data
Aug. 24, 2021 (JP) 2021-135966

(51) Int. Cl. A61L 24/10; A61L 26/00; C09J 189/00

(52) U.S. Cl. CPC A61L 24/104; A61L 26/0038; C09J 189/00

(57) ABSTRACT

The adhesive includes a first agent that includes a gelatin derivative in which a hydrophobic group is bound to gelatin through an imino group represented by a formula 1: GltnNH-R¹ and includes cyclodextrin; and a second agent that includes a crosslinking agent for the gelatin derivative. The cured product is resistant to swell in a physiological saline solution. In the formula 1, Gltn represents a residue of the gelatin, R¹ represents the hydrophobic group, and NH represents the imino group binding to the residue and the hydrophobic group.

### TECHNICAL FIELD

The present invention relates to an adhesive, a wound dressing material, an anti-adhesion material, a hemostatic material, a sealant, and a spray kit.

### BACKGROUND ART

Tissue adhesives using gelatin derivatives obtained by introducing hydrophobic groups into gelatin are known. Patent Literature 1 describes “a tissue adhesive that is applied to tissue by mixing an adhesive ingredient containing an aqueous solution of fish-derived gelatin and a curing ingredient containing an aqueous solution of a water-soluble crosslinking molecule, wherein the molecular main chain of the water-soluble crosslinking molecule includes an amide bond, an ethylene glycol unit, or a sugar chain and also includes two or more active ester groups, an acid anhydride, or an aldehyde group as a feature”.

### CITATION LIST

### Patent Literature

Patent Literature 1: International Publication No. WO 2014/112208

### SUMMARY OF INVENTION

### Technical Problem

The above tissue adhesive has excellent adhesion to biotissue and also has excellent biocompatibility and is therefore greatly expected to be applied in the future. However, the present inventor has obtained knowledge that the cured product swells in a physiological saline solution in some cases, and there is room for improvement.

In general, since the site to which a surgical adhesive is applied is rich in fluid such as an exudate and blood, if the adhesive (and a cured product thereof) absorbs the fluid and thereby swells, the adhesive may peel off from the application site, for example, a closed wound area.

Even if the adhesive does not peel off, swelling may cause shearing stress in the interface between the cured product and the biotissue, and it is also anticipated an increase of the burden on biotissue. Accordingly, it is preferable that the cured adhesive should be resistant to swell.

Accordingly, it is an object of the present invention to provide an adhesive of which it is difficult for the cured product to swell in a physiological saline solution. It is also an object of the present invention to provide a wound dressing material, an anti-adhesion material, a hemostatic material, a sealant, and a spray kit.

### Means of Solving Problem

The present inventors have intensively studied to achieve the above-mentioned objects and as a result has found that the objects can be achieved by the following configurations.

[1] An adhesive comprising a first agent that includes a gelatin derivative in which a hydrophobic group is bound to gelatin through an imino group represented by the formula 1 described later and includes cyclodextrin and comprising a second agent that includes a crosslinking agent for the gelatin derivative.

Since the cured product of the adhesive is resistant to swell in a physiological saline solution, even after applied to biotissue, the adhesive is resistant to peel off from the biotissue and not likely to cause a stress at the interface thereof, and the burden on the adhesive-applied biotissue is likely to be decreased.

[2] The adhesive according to [1], wherein the introduction rate of the hydrophobic group in the first agent is 5.0 to 80.0 mol %.

The adhesive having an introduction rate within the numerical range above has a more excellent effect of the present invention.

[3] The adhesive according to [1] or [2], wherein the first agent includes a solvent, and the concentration of the gelatin derivative in the first agent is 0.010 to 0.300 g/mL.

The adhesive having a concentration of the gelatin derivative within the numerical range above has a more excellent effect of the present invention.

[4] The adhesive according to [3], wherein the concentration is greater than 0.050 g/mL and less than 0.150 g/mL.

When the concentration is greater than 0.050 g/mL, the obtained adhesive has more excellent pressure resistance strength, and when the concentration is less than 0.150 g/mL, the cured product is less likely to swell in a physiological saline solution and has more excellent pressure resistance strength.

[5] The adhesive according to [2], wherein the introduction rate is 10.0 to 50.0 mol %.

When the introduction rate is 10.0 mol % or more, the cured product is less likely to swell in a physiological saline solution and has more excellent pressure resistance strength, and when the rate is 50.0 mol % or less, the adhesive has more excellent pressure resistance strength.

[6] The adhesive according to any one of [1] to [5], wherein the hydrophobic group includes a linear or branched alkyl group having 1 to 20 carbon atoms (“alkyl group A1”).

When the hydrophobic group includes the alkyl group A1, the adhesive has more excellent adhesive strength to biotissue, and the viscosity of the first agent is more likely to decrease.

[7] The adhesive according to [6], wherein R¹ in the formula 1 described later includes a linear alkyl group having 7 to 12 carbon atoms (hereinafter, also referred to as “alkyl group A2”).

When R¹ in the formula 1 described later includes the alkyl group A2, the adhesive has further excellent adhesive strength to biotissue, and the viscosity of the first agent is further likely to decrease.

[8] The adhesion according to any one of [1] to [7], wherein the cyclodextrin is α-cyclodextrin or a derivative thereof.

Since α-cyclodextrin easily includes an alkyl group (in particular, the alkyl group A1 and the alkyl group A2), the viscosity of the consequently obtained first agent is more likely to decrease. The cured product is more resistant to swell in a physiological saline solution.

[9] The adhesive according to any one of [1] to [8], wherein the molar-based ratio of the content of cyclodextrin to the content of the hydrophobic group (also referred to as “Cy/HBic”) in the first agent is 0.1 or more.

In the first agent of an adhesive of which Cy/HBic is within the range above, the amount of cyclodextrin with respect to the hydrophobic group of the gelatin derivative is likely to be sufficient, and as a result, the viscosity of the first agent of the obtained adhesive is more likely to decrease, and the cured product is more resistant to swell in a physiological saline solution.

The adhesive according to [9], wherein the molar-based ratio is greater than 2.6 and 10 or less. The first agent of an adhesive of which Cy/HBic is within the range above is likely to have a lower viscosity and is suitable as a spraying adhesive.

[11] The adhesive according to any one of [1] to [10], wherein the crosslinking agent is a compound including at least two active ester groups.

When the curable group of the crosslinking agent is an active ester group, since the active ester group easily reacts selectively with a primary amino group in mild conditions, more excellent characteristics as a room temperature curing type adhesive are obtained.

[12] The adhesive according to any one of [1] to [11], wherein the gelatin is cold-water fish gelatin.

Since cold-water fish gelatin has excellent fluidity at biological temperature, an adhesive of which the raw material gelatin is cold-water fish gelatin is likely to have a lower viscosity.

[13] A wound dressing material comprising the adhesive according to any one of [1] to.

[14] An anti-adhesion material comprising the adhesive according to any one of [1] to [12].

[15] A hemostatic material comprising the adhesive according to any one of [1] to.

[16] A sealant comprising the adhesive according to any one of [1] to [12].

[17] A spray kit comprising the adhesive according to any one of [1] to and a sprayer for the adhesive.

### Advantageous Effects of Invention

According to the present invention, it is possible to provide an adhesive of which the cured product is resistant to swell in a physiological saline solution. In addition, according to the present invention, it is also possible to provide a wound dressing material, an anti-adhesion material, a hemostatic material, a sealant (sealing material), and a spray kit.

### BRIEF DESCRIPTION OF DRAWINGS

FIG. 1 is an explanatory drawing of component parts of a sprayer included in a spray kit according to an embodiment of the present invention.

FIG. 2 is an explanatory drawing of an assembled spray kit.

FIG. 3 is a flow chart explaining a method for applying an adhesive to target tissue by a spray kit.

### DESCRIPTION OF EMBODIMENTS

The present invention will now be described in detail.

The description of the configuration requirements described below may be made based on representative embodiments of the present invention, and the present invention is not limited to the embodiments.

In the present specification, a numerical range expressed using “X to Y” means a range that includes the numerical values written before and after “to” as lower and upper limits.

In the description of groups (atomic groups) in the present specification, descriptions that do not indicate substitution and unsubstitution include those not having a substituent as well as those having a substituent, within a range not impairing the effects of the present invention. For example, “alkyl group” encompasses not only an alkyl group not having a substituent (unsubstituted alkyl group) but also an alkyl group having a substituent (substituted alkyl group). This also applies to each compound.

### [Adhesive]

The adhesive according to an embodiment of the present invention comprises a first agent including a gelatin derivative in which a hydrophobic group is bound to gelatin through an imino group represented by the formula 1 described later and cyclodextrin and comprises a second agent including a crosslinking agent for the gelatin derivative. Hereinafter, ingredients included in the adhesive (hereinafter, also referred to as “the present adhesive”) according to the present embodiment will be described.

### <First Agent>

The first agent according to the present embodiment includes a gelatin derivative and cyclodextrin and may further include a solvent.

The first agent is mixed with the second agent described later, the gelatin derivative is crosslinked by a crosslinking agent, and a skeleton of the cured product is formed. The curing reaction is typically a reaction between a primary amino group of hydrophobically modified gelatin and a crosslinkable group (typically, e.g., an active ester group) of the second agent.

At this point, the content of the first agent in the present adhesive is, in relationship with the content of the crosslinkable group of the second agent described later, preferably adjusted such that the amount of the crosslinkable group in the second agent is 0.1 to 3.0 equivalents per equivalent of the amino group in the first agent, more preferably 0.2 to 2.0 equivalents, further preferably 0.3 to 1.5 equivalents, and particularly preferably 0.3 to 0.8 equivalents.

### (Gelatin Derivative)

The content of the gelatin derivative in the first agent is not particularly limited, but the concentration of the gelatin derivative in the first agent (gelatin derivative/solvent, round off at the fourth decimal place) is preferably 0.010 to 0.300 g/mL, more preferably greater than 0.050 g/mL, further preferably 0.075 g/mL or more, particularly preferably greater than 0.075 g/mL and preferably 0.150 g/mL or less, more preferably less than 0.150 g/mL, and particularly preferably 0.100 g/mL or less.

When two or more gelatin derivatives are used in combination, the total amount thereof is preferably within the above numerical range.

When the concentration of the gelatin derivative is greater than 0.050 g/mL, an adhesive with more excellent pressure resistance strength is obtained. When the concentration of the gelatin derivative is less than 0.150 g/mL, the cured product is less likely to swell, and an adhesive with more excellent pressure resistance strength is obtained. When the concentration of the gelatin derivative is 0.075 g/mL or more, an adhesive with further excellent pressure resistance strength is obtained. When the concentration of the gelatin derivative is 0.100 g/mL or less, the cured product is further less likely to swell, and an adhesive with further excellent pressure resistance strength is obtained.

The gelatin derivative is a gelatin derivative in which a hydrophobic group is bound to gelatin through an imino group (i.e., —NH—) and is represented by a formula 1: Formula 1: GltnNH-R¹.

In the formula 1, Gltn represents a gelatin residue, and NH represents an imino group binding to the gelatin residue and the hydrophobic group.

In the formula 1, R¹ represents a hydrophobic group. The hydrophobic group is not particularly limited, but is preferably a group including a hydrocarbon group having 1 to 20 carbon atoms.

Here, the group including a hydrocarbon group having 1 to 20 carbon atoms means a hydrocarbon group itself having 1 to 20 carbon atoms and a group including a hydrocarbon group having 1 to 20 carbon atoms and a linker group.

That is, when L is a single bond or a divalent linker group and R²¹ is a hydrocarbon group having 1 to 20 carbon atoms, the hydrophobic group is preferably a group represented by *-L-R²¹, where * represents a binding site.

Examples of the divalent linker group as L include —C(O)—, —C(O)O—, —OC (O)—, —O—, —S—, —N(R)— (R represents a hydrogen atom or a monovalent organic group (preferably hydrocarbon group having 1 to 20 carbon atoms)), an alkylene group (preferably an alkylene group having 2 to 10 carbon atoms), an alkenyl group (preferably an alkenyl group having 2 to 10 carbon atoms), and combination thereof. In particular, —O—, —C(O)—, and —C(O)O— are preferable.

When L includes a carbon atom, the total number of carbon atoms of L and R²¹ is preferably 1 to 20, more preferably 4 to 18, further preferably 6 to 14, and particularly preferably 7 to 12.

Examples of the hydrocarbon group having 1 to 20 carbon atoms include a chain hydrocarbon group having 1 to 20 carbon atoms, an alicyclic hydrocarbon group having 3 to 20 carbon atoms, an aromatic hydrocarbon group having 6 to 14 carbon atoms, and a group of combination thereof.

Examples of the chain hydrocarbon group having 1 to 20 carbon atoms include linear and branched alkyl groups, and preferred is a linear hydrocarbon group.

Examples of the linear or branched alkyl group include:
an alkyl group having one carbon atom, i.e., a methyl group;
an alkyl group having two carbon atoms, i.e., an ethyl group;
alkyl groups having 3 carbon atoms, i.e., a propyl group and an isopropyl group;
alkyl groups having 4 carbon atoms, i.e., a butyl group, an isobutyl group, a tert-butyl group, and a see-butyl group;
alkyl groups having 5 carbon atoms, i.e., a pentyl group, a 1-methylbutyl group, a 2-methylbutyl group, a 3-methylbutyl group, a 1,1-dimethylpropyl group, a 2,2-dimethylpropyl group, and a 1-ethylpropyl group;
alkyl groups having 6 carbon atoms, i.e., a hexyl group, a 1-methylpentyl group, a 2-methylpentyl group, a 3-methylpentyl group, a 4-methylpentyl group, a 1,1-dimethylbutyl group, a 1,2-dimethylbutyl group, a 1,3-dimethylbutyl group, a 1,4-dimethylbutyl group, a 2,3-dimethylbutyl group, a 2,2-dimethylbutyl group, a 3,3-dimethylbutyl group, a 1-ethylbutyl group, a 2-ethylbutyl group, a 1-ethyl-2-methyl-propyl group, and a 1,1,2-trimethylpropyl group;
alkyl groups having 7 carbon atoms, i.e., a heptyl group, a 1-methylhexyl group, a 2-methylhexyl group, a 3-methylhexyl group, a 4-methylhexyl group, a 5-methylhexyl group, a 1,1-dimethylpentyl group, a 2,2-dimethylpentyl group, a 3,3-dimethylpentyl group, a 4,4-dimethylpentyl group, a 1,2-dimethylpentyl group, a 1,3-dimethylpentyl group, a 1,4-dimethylpentyl group, a 2,3-dimethylpentyl group, a 2,4-dimethylpentyl group, a 3,4-dimethylpentyl group, a 1-ethylpentyl group, a 2-ethylpentyl group, a 3-ethylpentyl group, a 1,2,2-trimethylbutyl group, a 1,1,2-trimethylbutyl group, a 1,3,3-trimethylbutyl group, a 1,1,3-trimethylbutyl group, a 2,2,3-trimethylbutyl group, and a 2,3,3-trimethylbutyl group;
alkyl groups having 8 carbon atoms, i.e., an octyl group, a 1-methylheptyl group, a 2-methylheptyl group, a 3-methylheptyl group, a 4-methylheptyl group, a 5-methylheptyl group, a 6-methylheptyl group, a 1-ethylhexyl group, a 2-ethylhexyl group, a 3-ethylhexyl group, a 4-ethylhexyl group, a 1-propylpentyl group, a 2-propylpentyl group, a 1,1-dimethylhexyl group, a 2,2-dimethylhexyl group, a 3,3-dimethylhexyl group, a 4,4-dimethylhexyl group, a 5,5-dimethylhexyl group, a 3-ethyl-3-methylpentyl group, a 1,1-diethylbutyl group, a 2,2-diethylbutyl group, a 1,1,2,2-tetramethylbutyl group, a 1,1,3,3-tetramethylbutyl group, a 2,2,3,3-tetramethylbutyl group, and a 1,1-dimethyl-2-ethylbutyl group;
alkyl groups having 9 carbon atoms, i.e., a nonyl group, a 2-methyloctyl group, a 3-methyloctyl group, a 4-methyloctyl group, a 2,2-dimethylheptyl group, a 2,3-dimethylheptyl group, a 2,4-dimethylheptyl group, a 2,6-dimethylheptyl group, a 3,3-dimethylheptyl group, a 3,4-dimethylheptyl group, a 3,5-dimethylheptyl group, a 4,4-dimethylheptyl group, a 3-ethylheptyl group, a 4-ethylheptyl group, a 2,2,3-trimethylhexyl group, a 2,2,4-trimethylhexyl group, a 2,2,5-trimethylhexyl group, a 2,3,3-trimethylhexyl group, a 2,3,4-trimethylhexyl group, a 2,3,5-trimethylhexyl group, a 2,4,4-trimethylhexyl group, a 3,3,4-trimethylhexyl group, a 2-methyl-3-ethylhexyl group, a 3-methyl-3-ethylhexyl group, a 3-ethyl-4-methylhexyl group, a 3-ethyl-5-methylhexyl group, a 2,2,3,3-tetramethylpentyl group, a 2,2,3,4-tetramethylpentyl group, a 2,2,4,4-tetramethylpentyl group, a 2,3,3,4-tetramethylpentyl group, a 2,2-dimethyl-3-ethylpentyl group, a 2,3-dimethyl-3-ethylpentyl group, a 2,4-dimethyl-3-ethylpentyl group, and a 3,3-diethylpentyl group; and
alkyl groups having 10 carbon atoms, i.e., a decyl group, a 2-methylnonyl group, a 2-ethyloctyl group, a 2-propylheptyl group, and a 2-butylhexyl group.

In addition to the above, examples of the linear or branched alkyl group include an undecyl group, a dodecyl group, a tridecyl group, a tetradecyl group, a pentadecyl group, a hexadecyl group, a heptadecyl group, a hexadecyl group, a heptadecyl group, an octadecyl group, a nanodecyl group, and an icosyl group.

Examples of the alicyclic hydrocarbon group having 3 to 20 carbon atoms include a cyclopropyl group, a cyclopentyl group, a cyclohexyl group, an adamantyl group, and a norbornyl group.

The aromatic hydrocarbon group having 6 to 14 carbon atoms is not particularly limited, and examples thereof include a phenyl group, a tolyl group, and a naphthyl group.

The group of combination of the above is not particularly limited, and examples thereof include aralkyl groups having 6 to 12 carbon atoms, such as a benzyl group, a phenethyl group, a naphthylmethyl group, and a naphthylethyl group.

R¹ may be a group represented by the following formula. In the following structural formula, * represents a binding site.

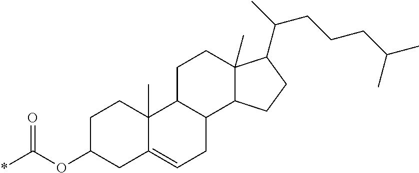 [SMILES: *C(=O)OC1CCC2(C)C(=CCC3C2CCC2(C)C(C(C)CCCC(C)C)CCC32)C1]

In the formula 1, the nitrogen atom (N) that directly binds to the residue of gelatin is mainly derived from the E-amino group of lysine (Lys) in the gelatin. The NH structure of the formula 1 can be detected by, for example, a band near 3300 cm-1 in an FT-IR (Fourier transform infrared spectrometer) spectrum.

From the viewpoint of obtaining an adhesive having a more excellent effect of the present invention, the gelatin derivative is preferably a gelatin derivative represented by a formula 11:

Formula 11: GltnNH-CHR²R³.

In the formula 11, R² represents a hydrocarbon group having 1 to 19 carbon atoms, and specific examples thereof include the same groups as the hydrocarbon groups having 1 to 19 carbon atoms among the hydrocarbon groups mentioned as R¹, and the same applies to preferred embodiments.

R³ represents a hydrocarbon group having 1 to 19 carbon atoms or a hydrogen atom and is preferably a hydrogen atom. The total number of carbon atoms of R² and R³ is not particularly limited and is preferably 2 to 19, more preferably 3 to 17, further preferably 5 to 13, and particularly preferably 6 to 11.

The introduction rate of the hydrophobic group in the first agent is not particularly limited and is preferably 5.0 to 80.0 mol % and more preferably 10.0 mol % or more and more preferably 50.0 mol % or less.

The introduction rate of the hydrophobic group is the value defined as the (content of imino group)/[(content of imino group)+(content of amino group)] in the first agent, and is the value that is obtained by quantify the amino group by a 2,4,6-trinitrobenzenesulfonic acid method.

The “introduction rate of the hydrophobic group in the first agent” is synonymous with the introduction rate of the hydrophobic group in the gelatin derivative, when the first agent does not contain gelatin other than the gelatin derivative (for example, raw material gelatin).

In contrast, for example, when the first agent includes a gelatin derivative and raw material gelatin described later (in other words, when the first agent is a mixture including both a gelatin derivative and a raw material gelatin), the introduction rate of the hydrophobic group is a value reflecting the total amount of amino groups possessed by the gelatin derivative and the raw material gelatin. Specifically, the introduction rate of the hydrophobic group in the first agent can also be calculated by the following expression A:

$$Expression A : introduction rate of hydrophobic group in first agent = Ha \times Ma / ( Ma + Mb ) .$$

In the expression, Ma and Mb represent the mass-based contents of the gelatin derivative and the gelatin in the first agent, respectively, and Ha represents the introduction rate of the hydrophobic group in the gelatin derivative.

When the introduction rate of the hydrophobic group in the first agent is 10.0 mol % or more, the cured product is less likely to swell, and when the rate is 50.0 mol % or less, an adhesive with more excellent pressure resistance strength is obtained.

Method for manufacturing gelatin derivative

The method for manufacturing the gelatin derivative is not particularly limited, and a known method can be used.

Examples of the method include a method of reacting aldehyde or ketone to the s-amino group possessed by gelatin to bind a hydrophobic group through a Schiff base and reducing the Schiff base to obtain a gelatin derivative. This method is described in, for example, paragraphs [0029] to [0031] of Japanese Unexamined Patent Application Publication No. 2019-216755.

According to the method above, a gelatin derivative in which a hydrophobic group is directly bound to a gelatin residue through an imino group (formula: GltnNH-R¹) is obtained. This hydrophobic group is derived from aldehyde or ketone.

Another example is a method of reacting an acid halide, a chloroformate compound, or the like to the E-amino group possessed by gelatin in the presence of a base such as triethylamine to obtain an amide. This method is described in, for example, paragraphs to of International Publication No. WO 2014/112208.

According to the method above, a gelatin derivative in which a hydrophobic group is bound to a gelatin residue through an amide bond (including an imino group) is obtained. This hydrophobic group is derived from an acid halide or a chloroformate compound.

Since the gelatin derivative precipitates by adding a large excess of a poor solvent, e.g., cold ethanol, to the reaction solution obtained above, a powdered gelatin derivative can be obtained by filtering and drying the precipitate. Before drying, the gelatin derivative may be washed with ethanol or the like.

The raw material gelatin (hereinafter, also referred to as “ORG gelatin”) that is used for manufacturing the gelatin derivative is typically gelatin in which a hydrophobic group has not been introduced (not derivatized gelatin).

The molecular weight of the ORG gelatin is not particularly limited and, in general, is preferably 10,000 to 300,000 weight average molecular weight. As one aspect, from the viewpoint of easily suppressing an allergic reaction by a living body, a molecular weight of less than 50,000 is preferable. From this point of view, the molecular weight of gelatin is preferably 45,000 or less and more preferably 40,000 or less. The lower limit is not particularly limited but is preferably 10,000 or more from the viewpoint that the cured product of the adhesive has more excellent mechanical strength.

As the ORG gelatin, any of gelatin that is naturally occurring or is obtained by chemical synthesis, fermentation, genetic recombination, or the like can be used without any limitation. In particular, naturally occurring gelatin is preferable. Examples of the naturally occurring gelatin include those derived from mammals such as a cow and a pig and those derived from fish such as sea bream, sturgeon, salmon, and cod.

When the present adhesive is used in a liquid form, it is preferable to have excellent fluidity at the operating temperature (e.g., biological temperature) from the viewpoint of the handling property.

From this point of view, the ORG gelatin is preferably fish-derived gelatin, in particular, gelatin derived from cold-water fish such as salmon and pollack is preferable.

The “use as a liquid” is when one of or both the first agent and the second agent is a liquid containing a solvent or when both the first agent and the second agent are solid and are mixed with a solvent when using.

In the fish-derived gelatin, in particular, cold-water fish gelatin, the number of the units derived from hydroxyproline and/or the number of the units derived from proline per 1,000 amino acids as the constitutional units are preferably 80 or less and 110 or less, respectively. Gelatin with these conditions has more excellent fluidity at ordinary temperature and therefore gives an adhesive with an excellent handling property, when used in the first agent (as a raw material of a gelatin derivative and/or an additive).

The ORG gelatin may be any of acid-treated gelatin and alkali-treated gelatin. The first agent may contain two or more different types of gelatin as the ORG gelatin. Two or more different types means that one or more of the origin, molecular weight, treatment method, and so on are different.

### (Cyclodextrin)

Cyclodextrin is a cyclic compound in which D-glucose units are linked into a ring by x-1,4-glycosidic bonds and is manufactured by applying an enzyme such as cyclodextrin glucaonotransferase to starch and/or starch hydrolysate.

The content of the cyclodextrin in the first agent is not particularly limited, but the concentration of the cyclodextrin in the first agent (cyclodextrin/solvent, round off at the fourth decimal place) is preferably 0.001 to 0.200 g/mL and more preferably 0.003 to 0.150 g/mL.

When two or more types of cyclodextrin are used in combination, the total amount thereof is preferably within the above numerical range.

From the viewpoint of obtaining an adhesive with more excellent effects of the present invention, the molar-based ratio of the content of cyclodextrin to the content of the hydrophobic group in the first agent (cyclodextrin/hydrophobic group, “Cy/HBic”) is preferably 0.1 or more, more preferably 1.0 or more, further preferably greater than 1.0, particularly preferably greater than 2.0, and most preferably greater than 2.5 and preferably 10.0 or less, more preferably 8.0 or less, and further preferably 6.0 or less.

When two or more types of cyclodextrin are used in combination, the total amount thereof is preferably within the above numerical range.

When Cy/HBic is greater than 1.0, the viscosity of the first agent is more likely to decrease, and the adhesive is preferable for spray. When Cy/HBic is greater than 2.0, the viscosity is further likely to decrease, and when Cy/HBic is greater than 2.5, the above tendency is particularly significant.

It is inferred that cyclodextrin also includes a gelatin residue other than the hydrophobic group and that the concentration of the cyclodextrin in the first agent has a negative correlation with the viscosity of the first agent. From the viewpoint that the effect of viscosity reduction on the added cyclodextrin is more significant, as one aspect, Cy/HBic is preferably 10.0 or less, more preferably 8.0 or less, and further preferably 6.0 or less.

As the cyclodextrin, for example, cyclodextrin in which the number of glucose constituting the cyclodextrin is 6 (α-type), 7 (β-type), or 8 (γ-type) can be used, and a derivative thereof can be also used (or in combination).

In particular, α-cyclodextrin or a derivative thereof is preferable because the size of the inner cavity thereof is suitable for including the hydrophobic group.

Examples of the derivative of α-cyclodextrin include methyl-α-cyclodextrin, butyl-α-cyclodextrin, 2-hydroxypropyl-α-cyclodextrin, acetyl-α-cyclodextrin, succinyl-α-cyclodextrin, glucosyl-α-cyclodextrin, maltosyl-α-cyclodextrin, α-cyclodextrin carboxymethyl ether, phosphate α-cyclodextrin, and carboxymethyl-x-cyclodextrin.

Examples of the derivative of β-cyclodextrin include methyl-β-cyclodextrin (MBCD), (2-hydroxypropyl)-β-cyclodextrin (HPBCD), carboxymethyl-β-cyclodextrin, carboxymethyl-ethyl-β-cyclodextrin, diethyl-β-cyclodextrin, dimethyl-β-cyclodextrin, glucosyl-β-cyclodextrin, hydroxybutenyl-β-cyclodextrin, hydroxyethyl-β-cyclodextrin, maltosyl-β-cyclodextrin, random methyl-β-cyclodextrin, sulfobutyl ether β-cyclodextrin, 2-selenium crosslinked β-cyclodextrin, and 2-tellurium crosslinked β-cyclodextrin.

Examples of the derivative of γ-cyclodextrin include 2-hydroxyethyl-γ-cyclodextrin, 2-hydroxypropyl-γ-cyclodextrin, butyl-γ-cyclodextrin, 3A-amino-3A-deoxγ-(2AS, 3AS)-γ-cyclodextrin, mono-2-O-(p-toluenesulfonyl)-γ-cyclodextrin, mono-6-O-(p-toluenesulfonyl)-γ-cyclodextrin, mono-6-O-mesitylenesulfonyl-γ-cyclodextrin, octakis (2,3,6-tri-O-methyl)-γ-cyclodextrin, octakis (2,6-di-O-phenyl)-γ-cyclodextrin, octakis (6-O-t-butyldimethylsillyl)-γ-cyclodextrin, and octakis (2,3,6-tri-O-acetyl)-γ-cyclodextrin.

### (Solvent)

The first agent may further include a solvent. Examples of the solvent include aqueous solvents, and as the aqueous solvent, for example, ultrapure water; a physiological saline solution; an inorganic salt buffer solution, such as borate, phosphate, and carbonate buffer solutions; or a mixture thereof can be used. In particular, the aqueous solvent is preferably a borate buffer solution with a pH of 8 to 13 and more preferably a borate buffer solution with a pH of 9 to 12. The aqueous solvent is preferably used in an amount such that the solid content of the first agent is 0.050 to 0.800 g/mL.

The first agent may include, in addition to the above, the ORG gelatin described as raw material gelatin.

### <Second Agent>

The second agent includes a crosslinking agent for the gelatin derivative. The second agent may include a solvent.

### (Crosslinking Agent)

The crosslinking agent is typically a compound including at least two substituents (crosslinkable groups) that can react with the primary amino group of the gelatin derivative in one molecule.

When the first agent includes the ORG gelatin, the primary amino group of the ORG gelatin also reacts by the crosslinking agent.

The crosslinkable group possessed by the crosslinking agent is not particularly limited, but is preferably an active ester group (activated ester group) from the viewpoint of easily reacting selectively with the primary amino group (typically, derived from the gelatin derivative) in the first agent under mild conditions. That is, the crosslinking agent is preferably a compound having at least two active ester groups in one molecule.

Examples of such a crosslinking agent include polybasic acid activated by N-hydroxysuccinimide or N-hydroxysulfosuccinimide.

In addition to the above, as the crosslinking agent, for example, Genipin, an aldehyde compound, an acid anhydride, dithiocarbonate, or diisothiocyanate can be used.

Examples of the polybasic acid include tartaric acid, citric acid, malic acid, glutaric acid, glutamic acid, aspartic acid, oxaloacetic acid, cis-aconitic acid, 2-ketoglutaric acid, polytartaric acid, polycitric acid, polymalic acid, polyglutamic acid, polyaspartic acid, carboxymethylated dextrin, carboxymethylated dextran, carboxymethylated starch, carboxymethylated cellulose, carboxymethylated chitosan, and carboxymethylated pullulan.

As the crosslinking agent, for example, disuccinimidyl glutarate (DSG), disuccinimidyl suberate (DSS), and disuccinimidyl tartrate (DST) can also be used.

In addition, the crosslinking agent is also preferably, for example, polyethylene glycol or a polybasic acid ester of polyethylene glycol ether in which at least one of carboxyl groups not reacting with polyethylene glycol in the polybasic acid has been converted into an active ester, for example, di (N-succinimidyl) 4,7,10,13,16-pentaoxanonadecanedioate and polyethylene glycol di (succinimidyl succinate) (SS-PEG-SS) represented by the following formula:

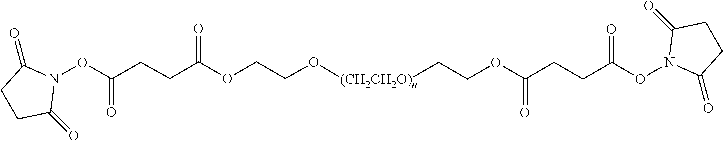

(n is a number making the number average molecular weight about 20,000); or pentaerythritol-polyethylene glycol ether tetrasuccinimidyl glutarate (4S-PEG) represented by the following structure formula:

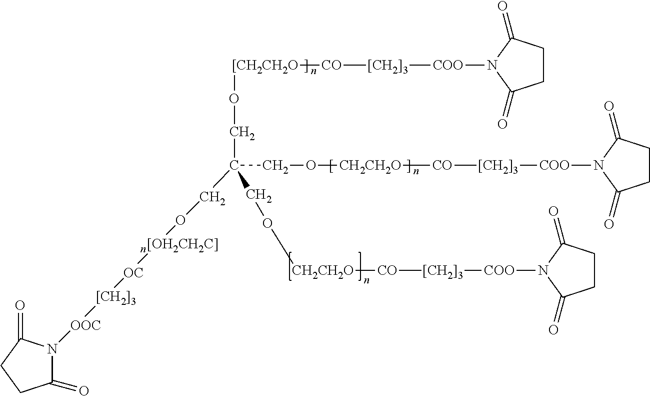

(n is a number making the Mw about 3,000 to 30,000, preferably 5,000 to 27,000, and more preferably 15,000 to 25,000).

Examples of the aldehyde compound include formyl group-introduced polysaccharides having two or more formyl group in one molecule, such as formyl group-introduced starch, formyl group-introduced dextran, formyl group-introduced dextrin, and formyl group-introduced hyaluronic acid.

Examples of the acid anhydride include glutaric anhydride, maleic anhydride, and succinic anhydride.

Examples of the diisothiocyanate include hexamethylene diisothiocyanate.

The crosslinking agent is preferably activated polyethylene glycol polybasic acid ester or a formyl group-introduced polysaccharide and more preferably activated polyethylene glycol polybasic acid ester.

The content of the crosslinking agent in the second agent and the content of the second agent in the adhesive may be appropriately adjusted according to the content of the amino group in the first agent.

For example, the amount of the ester group activated by N-hydroxysuccinimide (active ester group) is preferably 0.1 to 3.0 equivalents, more preferably 0.2 to 2.0 equivalents, further preferably 0.3 to 1.5 equivalents, and particularly preferably 0.3 to 0.8 equivalents per equivalent of the amino group in the first agent.

The second agent may contain one crosslinking agent alone or may contain two or more crosslinking agents. When the second agent contains two or more crosslinking agents, the total content thereof is preferably within the above numerical range.

### (Solvent)

The second agent may include a solvent. The solvent is preferably an aqueous solvent.

As the aqueous solution, the aqueous solutions already described as those that may be included in the first agent can be used.

In particular, a phosphate buffer solution with a pH of 3 to 8 is preferable, and a phosphate buffer solution with a pH of 4 to 6 is more preferable.

The ionic strengths of both the aqueous solvents included in the first agent and the second agent are preferably adjusted such that the pH is about 8 to about 10 when the first agent and the second agent are mixed in the same volume.

For example, when the first agent and the second agent are mixed in the same volume, the pH can be adjusted within the above range by using a borate buffer solution having a pH of 9 and an ionic strength of 0.05 to 0.1 as the first agent and a phosphate buffer solution having a pH of 4 and an ionic strength of 0.01 to 0.03 as the second agent. The first agent and the second agent may be a borate buffer solution having a pH of 10 and an ionic strength of 0.05 to 0.1 and a phosphate buffer solution having a pH of 4 and an ionic strength of 0.01 to 0.07, respectively.

### <Additive>

The first agent and/or the second agent may further include various additives in amounts that do not impede the object of the present invention. Examples of the additive include a colorant, a pH adjuster, and a preservative and so on. For example, a colorant (e.g., brilliant blue) may be added to the first agent and/or the second agent so that the application site of the adhesive is easily recognized. The addition amount may be, for example, 10 to 100 μg/mL.

The adhesive (in particular, first agent) of the present invention has a low viscosity and is therefore preferably used in a form for application by discharging in a mist form using a sprayer. In addition, the viscosity of the adhesive can be easily increased by addition of a viscosity modifier, and also dripping of the coated layer before curing can be suppressed. Accordingly, the adhesive is excellent also in that the viscosity can be adjusted according to the application site and the purpose (since the initial viscosity is low, there is room for thickening).

The present adhesive includes a gelatin derivative having a hydrophobic group introduced into gelatin and may include “another” gelatin and/or “another” gelatin derivative, as long as including the gelatin derivative.

That is, when the first agent includes a derivative of Walleye pollack gelatin, the first agent may include, for example, the Walleye pollack gelatin, pig gelatin, and/or a pig gelatin derivative.

### [Method for Manufacturing Adhesive]

The present adhesive can be obtained by preparing the first agent and the second agent, separately. The methods for preparing the first agent and the second agent will now be described, respectively.

### <Method for Preparing First Agent>

The first agent can be manufactured by mixing a gelatin derivative, cyclodextrin, and as needed, other ingredients. On this occasion, it is preferable to include a step of including at least a part of the hydrophobic groups of the gelatin derivative in cyclodextrin (forming an inclusion compound).

The method for including the hydrophobic group of the gelatin derivative in cyclodextrin is not particularly limited, and a method of adding and mixing a gelatin derivative to a slurry prepared by adding water to cyclodextrin or a method of dissolving cyclodextrin and a gelatin derivative in a solvent and drying the solution can be used.

In the method of dissolving cyclodextrin and a gelatin derivative in a solvent and drying the solution, the obtained first agent is in a powder form. When this is used as a liquid first agent, an aqueous solvent such as a borate buffer solution may be added to the above powdery first agent. An additive may be added at this point as needed.

The obtained first agent can be filled into a predetermined container, for example, a dispenser made of plastic such as polypropylene. When the first agent is used as a tissue adhesive, an aqueous solution of the first agent is preferably filled into one of a double syringe type dispenser or the like that can mix two agents at the tip portion, which is used when applied to tissue.

### <Method for Preparing Second Agent>

The second agent includes a crosslinking agent. The crosslinking agent may be synthesized by a known method or may be a commercially available one. When the second agent is formed as a liquid, the crosslinking agent and an aqueous solvent for dissolving it, such as a phosphate buffer solution, may be mixed.

### <Application Method to Tissue>

The present adhesive can be applied to incision in various surgery operations, such as respiratory surgery, gastrointestinal surgery, cardiovascular surgery, neurosurgery, and oral surgery, and a skin wound.

A curing reaction occurs immediately by mixing the two agents to form a cured product. The temperature during the curing reaction is not particularly limited but is generally preferably 15° ° C. to 45° C. and more preferably 20° ° C. to 42° C. The curing time is not particularly limited, but sufficient adhesive strength and membrane strength are obtained in 1 to 60 minutes.

The present adhesive can be used as a wound dressing material for dressing the wound caused in biotissue. It also can be used as an anti-adhesion material for preventing postoperative adhesion.

The cured product of the present adhesive has both excellent tissue adhesion and flexibility and therefore can also be used as, for example, a hemostatic material for stopping bleeding from a vascular anastomotic site by application to the vascular anastomotic site.

In addition, the present adhesive has excellent pressure resistance strength when applied to tissue as described later and therefore can withstand blood pressure and follows the blood vessel pulsation due to the flexibility.

Since the cured product of the present adhesive has, in addition to the excellent adhesion, both excellent absorbability and biocompatibility, for example, the present adhesive can also be used as a sealant or the like, when suturing the dura mater, for filling the gap between the dura mater and the dura mater, the dure mater suture area, or the gap between the dura mater-forming material and the dura mater.

The method of using the present adhesive is not particularly limited, but is preferably a form of applying the present adhesive to an objective site (tissue) using a sprayer described later and forming a cured product (gel) on the tissue.

### [Spray Kit]

The spray kit according to an embodiment of the present invention is configured by including a sprayer, a first agent, and a second agent and is used for applying the adhesive obtained by mixing the first agent and the second agent to a subject such as biotissue in a mist form.

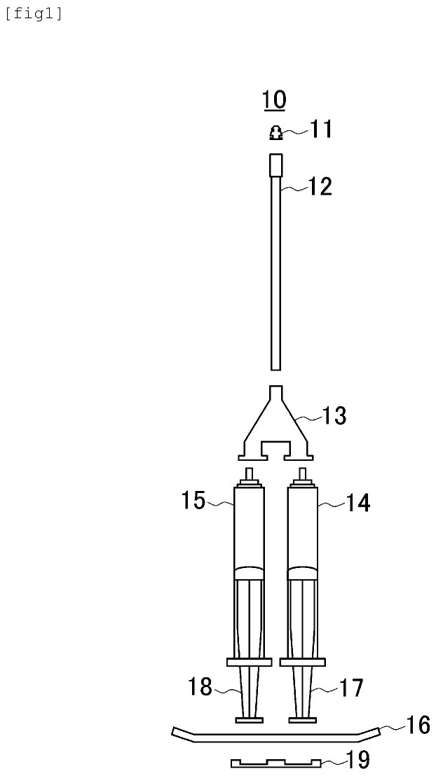

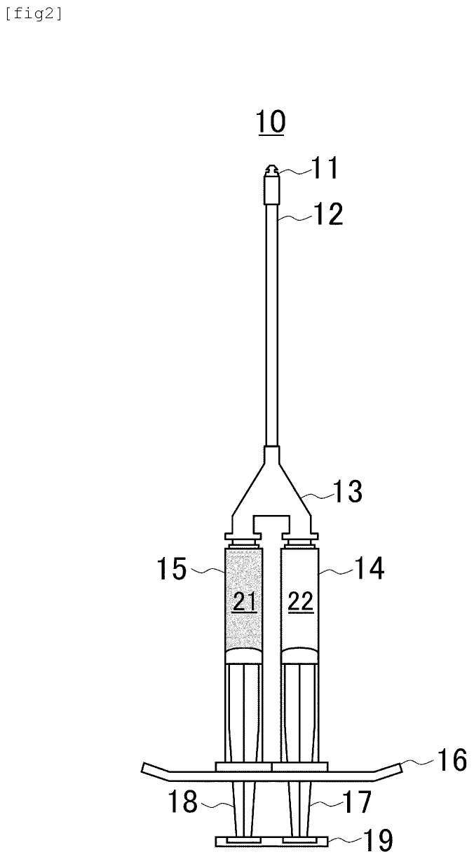

FIG. 1 is an explanatory drawing of component parts of a sprayer included in a spray kit, and FIG. 2 is an explanatory drawing of an assembled spray kit.

The sprayer 10 includes a first agent syringe consisting of an outer cylinder 15 and a plunger 18 with a gasket disposed at the tip and a second agent syringe consisting of an outer cylinder 14 and a plunger 17 with a gasket disposed at the tip. Typically, the same amount of the first agent 21 and the second agent 22 are injected into the syringes, respectively.

In FIG. 2, the first agent 21 is colored, and the second agent 22 is not colored. However, the second agent 22 may be colored, or both the agents need not be colored.

The outer cylinder 14 and the outer cylinder 15 are held with a syringe holder 16 in a restricted state so that they do not move, and an applicator 13 is fitted into the tips. Flow channels (not shown) for the first agent 21 and the second agent 22 are formed respectively in the inside of the applicator 13, the first agent and the second agent are extruded from the respective syringes and flow to the tip of the applicator 13 without mixing. The inner flow channels may be formed such that the first agent and the second agent are mixed with each other in the applicator.

A plunge cap 19 is fitted into the rear tips of the plunger 17 and the plunger 18. The two plungers 17 and 18 can be pushed as one unit by pushing this plunger cap 19 into the direction of the syringe holder 16, and the same amount of the first agent 21 and the second agent 22 in the syringes can be extruded.

The first agent and second agent extruded from the syringes and passed through the flow channels in the applicator 13 are discharged from the spray tip 11 in a mist form through the flow channel in the extender 12.

The sprayer 10 includes an extender 12 but need not include the extender 12. When the sprayer 10 does not include the extender 12, the spray tip 11 may be connected to the outlet of the applicator 13.

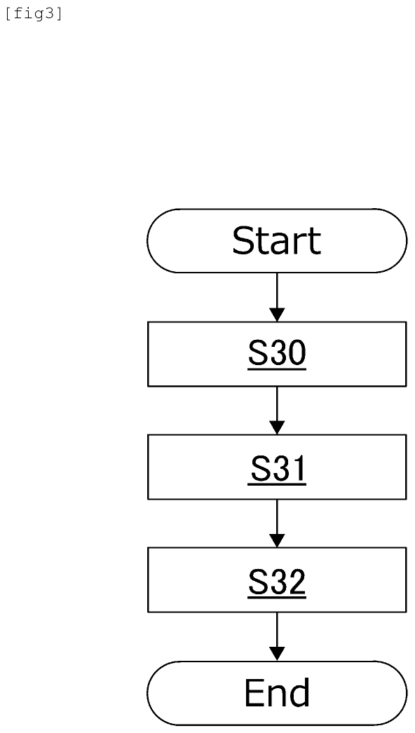

The method for using the spray kit will now be described. FIG. 3 is a flow chart explaining a method for applying an adhesive to target tissue by a spray kit.

First, in step S30, a first agent and a second agent are prepared respectively. The methods for preparing the first agent and the second agent are not particularly limited. In an example of the method, predetermined amounts of solvents are added to and mixed with, respectively, a first agent and a second agent not containing solvents.

More specifically, in one form, the spray kit includes vial bottles respectively containing the first agent and second agent in powder forms, and predetermined amounts of solvents are injected into the respective vial bottles to prepare a liquid first agent and a liquid second agent. These solvents may be injected in the first agent syringe and the second agent syringe, respectively, in advance.

Subsequently, in step S31, the prepared liquid first agent and second agent are respectively injected into the syringes to assemble a sprayer. Specifically, the agents prepared by mixing in the vial bottles are aspirated by the respective syringes, and a sprayer may be then assembled.

Subsequently, in step S32, the adhesive in a mist form is discharged from the spray tip by pushing a plunger cap. By this step, the adhesive is applied to target tissue and rapidly solidifies into gel.

The cured product of the adhesive obtained by mixing the above-described first agent and second agent has a characteristic of being resistant to swell in a physiological saline solution and therefore it is resistant to peel off even in an environment with a lot of moisture such as exudate and blood and has characteristics of being less likely to cause a stress at the interface and decreasing the burden on the biotissue.

### EXAMPLES

The present invention will now be described by examples but is not limited thereto.

(1) Preparation of gelatin derivative “18C10”

Ten grams of alkali-treated gelatin (Mw: 37,000, “beMatrix fish gelatin TA (trade name)”, manufactured by Nitta Gelatin Inc., hereinafter, referred to as “Org gelatin”) derived from Walleye pollack was added to and dissolved in 50 mL of an ultrapure water-ethanol mixture solvent in an eggplant-shaped flask immersed in an oil bath of 50° C. while stirring about 2 hours to prepare 20 mass % aqueous solution. Subsequently, picolineborane (manufactured by Junsei Chemical Co., Ltd.) equivalent to 1.5 times that of decanal added in the later state was added to the obtained aqueous solution, and decanal (manufactured by Tokyo Chemical Industry Co., Ltd.) equivalent to 0.5 times that of the amino group of gelatin (molar ratio of decanal to 1 mole of the amino group of gelatin) was then added thereto.

Subsequently, the eggplant-shaped flask is equipped with a reflux condenser, and the mixture was reacted while stirring at 55ºC for 18 hours. Subsequently, the reaction solution was dropwise added to 1 L of ethanol for reprecipitation. After stirring for 1 hour and then leaving in a freezer for 1 hour, filtration through a glass filter was performed. The filtration residue was added again to 1 L of ethanol in a beaker for reprecipitation, followed by stirring for 1 hour and then leaving in a freezer for 1 hour. Filtration through a glass filter was performed again, and the filtration residue was then dried in a vacuum drier at least overnight to obtain a gelatin derivative in which a decyl group (C10) as a hydrophobic group was introduced into the gelatin residue through an imino group at a yield of 98%.

The introduction rate of the decyl group in the obtained gelatin derivative was determined by the following method.

Org gelatin and a gelatin derivative were each dissolved in a water-DMSO (dimethyl sulfoxide) mixture solvent (volume ratio of 1:1, the same applies hereinafter) at 0.1 mass/vol %, and 100 μL of the solution was dispensed into a 48-well plate.

One hundred microliters of triethylamine (TEA, manufactured by Nacalai Tesque, Inc.) dissolved in a water-DMSO mixture solvent at 0.1 vol/vol % was added to the solution, followed by stirring with a plate shaker at 400 rpm for 1 minute. Furthermore, 100 μL of trinitrobenzene sulfonate (TNBS, manufactured by FUJIFILM Wako Pure Chemical Corporation) dissolved in a water-DMSO mixture solvent at 0.1 mass/vol % was added thereto, followed by stirring with a plate shaker at 400 rpm for 1 minute. The solution was shaded with aluminum foil, left to stand in an incubator of 37ºC for 2 hours, and taken out from the incubator, and 50 μL of HCl (6 mol/L) was added thereto to stop the reaction, followed by stirring with a plate shaker at 400 rpm for 1 minute. Subsequently, the solution was shaded and left to stand for 10 minutes, and the absorbance (Abs) at 340 nm was then measured with an spectrophotometer (manufactured by TECAN, Spark 10M-NMST). The absorbance of a blank sample that was different only in that it did not contain gelatin was subtracted from the measured absorbance, and the decyl group introduction rate in the gelatin derivative was determined to be 18.0 mol % by the following equation:

$$Introduction rate ( mol \% ) = [ Abs ( raw material$$

gelatin)-Abs (gelatin derivative)]/[Abs (Org gelatin)]×100.

The gelatin derivative obtained by the above method was defined as “18C10”.

“7.8C10” having a decyl group introduction rate of 7.8 mol % was obtained as in above except that the amount of decanal was 0.1 equivalents.

“31C10” having a decyl group introduction rate of 31.0 mol % was obtained as in above except that the amount of decanal was 1.0 equivalents.

“51C10” having a decyl group introduction rate of 51.0 mol % was obtained as in above except that the amount of decanal was 2.0 equivalents.

### (2) Preparation of First Agent

Hydrophobically modified gelatin and α-cyclodextrin were respectively weighed, and 0.075 mol/L of a borate buffer solution (pH 9.5) was added thereto to prepare a first agent. Table 1 shows the composition of the first agent in each Example. For example, in Example 1, 75 mg of a gelatin derivative (7.8C10) and 8 mg of α-cyclodextrin (also referred to as “oCD”) were dispersed in 1 mL of a borate buffer solution.

### (3) Preparation of Second Agent

As a crosslinking agent, pentaerythritol-polyethylene glycol ether tetrasuccinimidyl glutarate (“4S-PEG”, weight average molecular weight: 20,000, manufactured by NOF Corporation) was provided. This was dissolved in 0.01 mol/L of a phosphate buffer solution (pH 4.0) to prepare a second agent.

### (4) Preparation of Adhesive

W syringe manufactured by ADY Co., Ltd. was loaded with the first agent and the second agent such that (NHS ester of crosslinking agent)/(primary amino group in first agent) was 0.5 (50 mol %) based on the mole ratio. The agents were extruded when using and were mixed. In Table 1, it is shown as “NHS/amine”.

### <Evaluation>

### (Evaluation of Swelling Property)

The agents were discharged from the W syringe to produce gel (adhesive cured product) with a thickness of 1 mm, and the gel was punched with a punch of a diameter of 10 mm as a sample.

Subsequently, the sample (adhesive cured product) was transferred to a 50-mL centrifuge tube, and 50 mL of a physiological saline solution (containing 25 μ/mL of Acid Bule) was poured therein, followed by leaving in an incubator of 37° C.

The change in mass was determined from the mass M₀ of the sample measured immediately before immersion and the mass M₁ of the sample after immersed in a physiological saline solution for 24 hours by the following expression. The lower this value is, the less the sample swells, i.e., the sample is excellent as an adhesive to be applied to biotissue. The measurement results of the adhesives of Examples and Comparative Example are shown in Table 1.

$$Mass change ( Δ ) = \vert ( {M}_{1} - {M}_{0} ) / {M}_{0} \vert$$

### (Adhesion to Biotissue)

Adhesion (pressure resistance strength) was evaluated using Collagen Casing (manufactured by Nippi, Incorporated) as model tissue for evaluating the tissue adhesive strength in accordance with ASTM-F2392-04R.

A pinhole with a diameter of 3 mm was formed in Collagen Casing with a diameter of 30 mm, and the adhesive was applied thereto at a thickness of 1 mm. This was left to stand at room temperature (23° C. to 25° C.) for 10 minutes, and the pressure resistance strength was measured. The test was carried out 5 times, and the average was evaluated by the following criteria. The evaluation results of the adhesives of Examples and Comparative Example are shown in Table 1.

Evaluation criteria:
AA: a pressure resistance strength of 14.5 kPa or more;
A: a pressure resistance strength of 11.5 kPa or more and less than 14.5 kPa;
B: a pressure resistance strength of 8.5 kPa or more and less than 11.5 kPa;
C: a pressure resistance strength of 5.5 kPa or more and less than 8.5 kPa; and
D: a pressure resistance strength of less than 5.5 kPa.

### (Viscosity Measurement)

The viscosity of each of the first agents prepared above was measured at 25° C. using a vibration viscometer “Viscomate VM-100A” (manufactured by Sekonic Corporation). The results are shown in Table 1.

TABLE 1
Table 1

| | First agent | | | | | | | |
|---|---|---|---|---|---|---|---|---|
| | Gelatin derivative | | | | | Result | | |
| | Introduction | | α-CD | | Second | | Pressure | |
| | rate | Concentration | Concentration | | agent | Mass | resistance | Viscosity |
| | [mol %] | [g/mL] | [g/mL] | Cy/Hbic | NHS/amine | change | strength | [mPa · s] |
| Example 1 | 7.8 | 0.075 | 0.008 | 4.0 | 0.5 | 0.56 | C | 4.4 |
| Example 2 | 18.0 | 0.050 | 0.012 | 4.0 | 0.5 | 0.06 | C | 3.8 |
| Example 3 | 18.0 | 0.075 | 0.005 | 1.0 | 0.5 | 0.22 | A | 10.1 |
| Example 4 | 18.0 | 0.075 | 0.009 | 2.0 | 0.5 | 0.26 | A | 8.4 |
| Example 5 | 18.0 | 0.075 | 0.018 | 4.0 | 0.5 | 0.19 | A | 6.6 |
| Example 6 | 18.0 | 0.100 | 0.024 | 4.0 | 0.5 | 0.46 | AA | 10.5 |
| Example 7 | 18.0 | 0.150 | 0.036 | 4.0 | 0.5 | 0.94 | B | 30.2 |
| Example 8 | 31.0 | 0.050 | 0.020 | 4.0 | 0.5 | 0.04 | C | 4.5 |
| Example 9 | 31.0 | 0.075 | 0.030 | 4.0 | 0.5 | 0.10 | B | 6.5 |
| Example 10 | 31.0 | 0.100 | 0.040 | 4.0 | 0.5 | 0.21 | AA | 10.9 |
| Example 11 | 31.0 | 0.150 | 0.060 | 4.0 | 0.5 | 0.80 | B | 34.3 |
| Example 12 | 51.0 | 0.050 | 0.032 | 4.0 | 0.5 | 0.06 | D | 4.0 |
| Example 13 | 51.0 | 0.075 | 0.049 | 4.0 | 0.5 | 0.12 | C | 6.5 |
| Example 14 | 51.0 | 0.100 | 0.064 | 4.0 | 0.5 | 0.25 | B | 11.5 |
| Example 15 | 51.0 | 0.150 | 0.097 | 4.0 | 0.5 | 0.84 | C | 30.0 |
| Comparative | 7.8 | 0.150 | 0.000 | — | 0.5 | 1.17 | B | 15.2 |
| Example 1 | — | — | — | — | — | 1.12 | A | 1.4 |
| Reference | | | | | | | | |
| Example 1 | | | | | | | | |

In Table 1, Comparative Example 1 is the result measured using an adhesive prepared as in Example 1 except that the first agent did not contain α-cyclodextrin.

Reference Example 1 is the result measured as in above except that “DuraSeal (registered trademark)” was used as the adhesive. “DuraSeal” is an adhesive that is used by mixing a first agent containing a polyethylene glycol ester compound and a phosphate buffer solution and a second agent containing an amino acid solution.

As shown in Table 1, the adhesive of Example 1 consisting of a first agent containing a gelatin derivative and cyclodextrin and a second agent containing a crosslinking agent therefor is less likely to swell, compared to the adhesive of Comparative Example 1 not containing cyclodextrin and the adhesive of Reference Example 1, and the pressure resistance strength and the viscosity were both within practical ranges.

The adhesive of Example 10 in which the concentration of the gelatin derivative in the first agent was greater than 0.050 g/mL and less than 0.150 g/mL had more excellent pressure resistance strength compared to the adhesive of Example 8, and was less likely to swell and had more excellent pressure resistance strength compared to the adhesive of Example 11.

This tendency was the same regardless of the introduction rate of the hydrophobic group in the gelatin derivative. That is, the adhesive of Example 6 having an introduction rate of 18.0% had more excellent pressure resistance strength, compared to the adhesive of Example 2, and was less likely to swell and had more excellent pressure resistance strength, compared to the adhesive of Example 7. The adhesive of Example 14 having an introduction rate of 51.0% had more excellent pressure resistance strength, compared to the adhesive of Example 12, and was less likely to swell and had more excellent pressure resistance strength, compared to the adhesive of Example 15.

The adhesives of Example 3 and Example 9 in which the average introduction rates of the hydrophobic group in the first agents were 10.0% to 50.0% were less likely to swell and had more excellent pressure resistance strength, compared to the adhesive of Example 1, and had more excellent pressure resistance strength, compared to the adhesive of Example 13.

In the adhesive of Example 5 in which the Cy/HBic in the first agent was greater than 1.0, the viscosity of the first agent was lower than that of the adhesive of Example 3.

In the adhesive of Example 5 in which the Cy/HBic in the first agent was greater than 2.0, the viscosity of the first agent was lower than that of the adhesive of Example 4.

The adhesive of Example 6 in which the concentration of the gelatin derivative in the first agent was greater than 0.075 g/mL had more excellent pressure resistance strength, compared the adhesive of Example 5. This tendency was the same regardless of the introduction rate of the hydrophobic group in the gelatin derivative. That is, the adhesive of Example 10 had more excellent pressure resistance strength, compared to the adhesive of Example 9, and the adhesive of Example 14 had more excellent pressure resistance strength, compared to the adhesive of Example 13.

### INDUSTRIAL APPLICABILITY

The cured product of the adhesive of the present invention is less likely to swell and therefore resistant to peel off and less likely to cause a stress at the interface. Therefore, the burden on the target tissue is small, and the adhesive can be used as a tissue adhesive that is used in surgery operation and so on. In addition, the adhesive of the present invention can also be used as a wound dressing material that covers the wound area occurring by surgery operation or the like and promotes wound healing. The present adhesive applied to a damaged area after tissue resection works as a physical partition for preventing adhesion of surrounding tissue occurring during the repair process and can also be used as an anti-adhesion material.

The adhesive of the present invention is resistnat to swell, and the viscosity can be adjusted to a low level. Accordingly, the adhesive can be applied in a mist form by using a sprayer. Since the viscosity can also be easily adjusted by using a known thickener or the like, it can also be easily applied as an adhesive in the field where fixing after application is important.

### REFERENCE SIGNS LIST

10: sprayer, 11: spray tip, 12: extender, 13: applicator, 14: outer cylinder, 15: outer cylinder, 16: syringe holder, 17, 18: plunger, 19: plunger cap, 21: first agent, 22: second agent

1. An adhesive comprising:
a first agent that includes a gelatin derivative in which a hydrophobic group is bound to gelatin through an imino group represented by a formula 1: GltnNH-R′ and includes cyclodextrin; and
a second agent that includes a crosslinking agent for the gelatin derivative
in the formula 1, Gltn represents a residue of the gelatin, R′ represents the hydrophobic group, and NH represents the imino group binding to the residue and the hydrophobic group.

2. The adhesive according to claim 1, wherein
an introduction rate of the hydrophobic group in the first agent is 5.0 to 80.0 mol %.

3. The adhesive according to claim 1, wherein
the first agent includes a solvent, and
a concentration of the gelatin derivative in the first agent is 0.010 to 0.300 g/mL.

4. The adhesive according to claim 3, wherein
the concentration is greater than 0.050 g/mL and less than 0.150 g/mL.

5. The adhesive according to claim 2, wherein
the introduction rate is 10.0 to 50.0 mol %.

6. The adhesive according to claim 1, wherein
the hydrophobic group includes a linear or branched alkyl group having 1 to 20 carbon atoms.

7. The adhesive according to claim 6, wherein
the hydrophobic group includes a linear alkyl group having 7 to 12 carbon atoms.

8. The adhesive according to claim 1, wherein
the cyclodextrin is α-cyclodextrin or a derivative thereof.

9. The adhesive according to claim 1, wherein
a molar-based ratio of the content of the cyclodextrin to the content of the hydrophobic group in the first agent is 0.1 or more.

10. The adhesive according to claim 9, wherein
the molar-based ratio is greater than 2.6 and 10 or less.

11. The adhesive according to claim 1, wherein
the crosslinking agent is a compound including at least two active ester groups.

12. The adhesive according to claim 1, wherein
the gelatin is cold-water fish gelatin.

13. A wound dressing material comprising the adhesive according to claim 1.

14. An anti-adhesion material comprising the adhesive according to claim 1.

15. A hemostatic material comprising the adhesive according to claim 1.

16. A sealant comprising the adhesive according to claim 1.

17. A spray kit comprising the adhesive according to claim 1 and a sprayer of the adhesive.

18. The adhesive according to claim 2, wherein
the first agent includes a solvent, and
a concentration of the gelatin derivative in the first agent is 0.010 to 0.300 g/mL.

19. The adhesive according to claim 18, wherein
the concentration is greater than 0.050 g/mL and less than 0.150 g/mL.
